(19) United States
(12) Patent Application Publication
(10) Pub. No.: US 2025/0302986 A1
(43) Pub. Date: Oct. 2, 2025

(54) NOVEL PEPTIDE, USGAES THEREOF AND PHARMACEUTICAL COMPOSITION HAVING THE SAME

(71) Applicant: Taipei Medical University, Taipei (TW)

(72) Inventors: Chin-Wei Wang, Taipei (TW); Han-Chung Wu, Taipei (TW); Jing-Jie Yu, Taipei (TW); Ruei-Min Lu, Taipei (TW); Monika Kumari, Taipei (TW)

(21) Appl. No.: 18/806,827

(22) Filed: Aug. 16, 2024

(30) Foreign Application Priority Data
Mar. 27, 2024 (TW) 113111505

(51) Int. Cl. A61K 47/69; A61K 31/704; C07K 7/08

(52) U.S. Cl. CPC A61K 47/6911; A61K 31/704; C07K 7/08

(57) ABSTRACT

The present invention provides a novel peptide, usages thereof and pharmaceutical composition having the same. The novel peptide is a first peptide. The pharmaceutical composition comprises the novel peptide and drug carriers, each novel peptide is connected to the surface of the drug carrier by a linker. The amino acid sequence of the first peptide is shown in SEQ ID NO: 1. The first peptide can bind to CD80 protein, besides, the present invention proves that the novel peptide of the present invention achieves the efficiency of enhancing cell uptake.

### CROSS-REFERENCE TO RELATED APPLICATIONS

This application claims priority to Taiwan Application Serial Number 113111505, filed Mar. 27, 2024, which is herein incorporated by reference in its entirety.

### BACKGROUND OF THE INVENTION

The present invention relates to the field of peptides, more particularly to novel peptides that can bind to differentiation group 80, usages of the novel peptides and pharmaceutical compositions having the novel peptides.

### DESCRIPTION OF THE RELATED ART

Cluster of Differentiation 80 (CD80), also known as B7-1, is a protein present on the surface of immune cells and M1 macrophages such as dendritic cells. Presently, CD80 regulating T cells plays an important role in immune function. As an example, it is currently known that CD80 can bind to CD28 on T cells to promote the activation of T cell. In addition, CD80 can also bind to CTLA-4 on T cells to inhibit the activity of T cell.

Although it is known that CD80 is important for immune regulation, current research on developing peptides that can bind to the CD80 protein is still very limited, and research on using peptides that can bind to the CD80 protein to perform drug delivery is also very rare. Accordingly, products of utilizing peptides that bind to the CD80 protein for drug delivery are also very rare, and it causes such development to be stagnated in the field.

### SUMMARY OF THE INVENTION

According to the problems of prior arts, the objective of the present invention is that novel peptides can bind to CD80 protein and liposomes comprising the novel peptides.

An embodiment of the present invention provides a novel peptide, which amino acid sequence is shown in SEQ ID NO: 1.

An embodiment of the present invention provides the novel peptide, which is binding to CD80 protein, and the amino acid sequence of the novel peptide is shown in SEQ ID NO: 1.

An embodiment of the present invention provides a pharmaceutical composition comprising the novel peptide and a drug carrier, wherein the amino acid sequence of the novel peptide is shown in SEQ ID NO: 1 and cross-linking on the surface of the drug carrier.

In an embodiment of the present invention, the drug carrier is selected from the group consisting of: liposomes, microcells, nanoparticles, solid lipid nanoparticles, nanoemulsions, and combinations thereof.

In an embodiment of the present invention, the drug carrier is liposome.

In an embodiment of the present invention, the drug carrier contains a drug.

In an embodiment of the present invention, the drug is an anti-cancer drug.

In an embodiment of the present invention, the drug is Doxorubicin.

In an embodiment of the present invention, the novel peptide cross-linking on the surface of the drug carrier via a linker.

In an embodiment of the present invention, the linker is polyethylene glycol.

As a conclusion, the novel peptide of the present invention is able to bind to CD80 protein, and proves that combining the first peptide or the second peptide on a drug carrier does facilitate cellular uptake and future development of drug delivery.

It should be understood, however, that this summary may not contain all aspects and embodiments of the present disclosure, that this summary is not meant to be limiting or restrictive in any manner, and that the disclosure as disclosed herein will be understood by one of ordinary skill in the art to encompass obvious improvements and modifications thereto.

### BRIEF DESCRIPTION OF THE DRAWINGS

The features of the exemplary embodiments believed to be novel and the elements and/or the steps characteristic of the exemplary embodiments are set forth with particularity in the appended claims. The Figures are for illustration purposes only and are not drawn to scale. The exemplary embodiments, both as to organization and method of operation, may best be understood by reference to the detailed description which follows taken in conjunction with the accompanying drawings in which:

FIG. 1 illustrates experimental graphs of fluorescence intensities of each group analyzed by a fluorescence spectrometer;

FIG. 2 illustrates experimental graphs of percentages of cellular uptake of analyzing an LD-4-9 group and a control group; and

FIG. 3 illustrates experimental graphs of percentages of cellular uptake of analyzing an LD-4-7 group and the control group.

### DETAILED DESCRIPTION OF THE EMBODIMENTS

The present disclosure will now be described more fully hereinafter with reference to the accompanying drawings, in which exemplary embodiments of the disclosure are shown. This present disclosure may, however, be embodied in many different forms and should not be construed as limited to the embodiments set forth herein. Rather, these embodiments are provided so that this present disclosure will be thorough and complete, and will fully convey the scope of the present disclosure to those skilled in the art.

Certain terms are used throughout the description and following claims to refer to particular components. As one skilled in the art will appreciate, manufacturers may refer to a component by different names. This document does not intend to distinguish between components that differ in name but function. In the following description and in the claims, the terms “include/including” and “comprise/comprising” are used in an open-ended fashion, and thus should be interpreted as “including but not limited to”. “Substantial/substantially” means, within an acceptable error range, the person skilled in the art may solve the technical problem in a certain error range to achieve the basic technical effect.

The following description is of the best-contemplated mode of carrying out the disclosure. This description is made for the purpose of illustration of the general principles of the disclosure and should not be taken in a limiting sense. The scope of the disclosure is best determined by reference to the appended claims.

Moreover, the terms “include”, “contain”, and any variation thereof are intended to cover a non-exclusive inclusion. Therefore, a process, method, object, or device that includes a series of elements not only includes these elements, but also includes other elements not specified expressly, or may include inherent elements of the process, method, object, or device. If no more limitations are made, an element limited by “include a/an . . . ” does not exclude other same elements existing in the process, the method, the article, or the device which includes the element.

### <<Phage Display Screening>>

In ‘Phage Display Screening”, biopanning is applied via phage display technology. First, the G protein magnetic beads (product name: Dynabeads™ Protein G; manufacturer: Invitrogen) are added to mix with the phage peptide library (product name: PhD-12 Phage Display Peptide Library; manufacturer: New England Biolabs), thus the mixture is incubated for 1 hour at 4° C. ambient conditions. Then, a magnet is used to remove the G protein magnetic beads to exclude phages bound to the G protein magnetic beads in the phage peptide library, thereby obtaining a purified phage peptide library. Continuously, the bait carrier is added to the purified phage peptide library and incubated overnight at 4° C. ambient conditions. The bait carrier is magnetic beads immobilized with mouse CD80 recombinant protein (product name: Recombinant Mouse B7-1/CD80 Fc Chimera His-tag Protein, CF; manufacturer: R&D Systems; catalog number: 740-B1). Therefore, the phages in the purified phage peptide library that bind to the mouse CD80 recombinant protein will be bound to the bait carrier, and then the bait carrier will be separated using the magnet. Then the bait carrier is rinsed with cleaning solution to eliminate phages with weak binding force. The cleaning solution is phosphate buffered saline (PBS) containing 0.1% (v/v) polysorbate-20 (Tween 20). Then, the bait carrier is mixed with 1 mL of log-phase ER2738 E-Coli in a shaker at 85 rpm and incubated at 37° C. for 30 minutes, and then the bait carrier is removed by the magnet, and the eluent remains, and the 50 microliter (L) of the eluate is taken to measure the titer. Before measuring the titer, first preparing the top agar and solid LB culture dish (Luria-Bertani agar plate) is a must, and storing them in a 4° C. refrigerator. The preparation method of the top agar is as follows of: adding 10 grams of LB medium powder (product name: LB Broths; manufacturer: Athena Enzyme Systems™; catalog number: 0103) per 0.5 liters of pure water, and then adding 3.5 grams of agarose powder (product name: SeaPlaque™ Agarose; Manufacturer: Lonza Bioscience; Cat. No.: 50101) for being sterilized in an autoclave, cooled and solidified, and then heated in a microwave oven before use to dissolve it into a liquid state. The solid LB culture dish configuration method is as follows of: adding 10 grams of LB medium powder (product name: LB Broths; manufacturer: Athena Enzyme Systems™; Cat. No.: 0103) per 0.5 liters of pure water, and then adding 7.5 grams of selective agar powder (product name: Select Agar™; manufacturer: Invitrogen; Cat. No.: 30391-023) for being sterilized in an autoclave, and then adding 500 μL of IPTG/X-GAL, which formula is as follows of in 25 mL of dimethylformamide (product name: 5-Bromo-4-chloro-3-indolyl β-D-galactopyranoside; manufacturer: UNI-ONWARD; Cat. No.: UR-XGAL-2G), adding 1.25 grams of isopropyl sulfated galactose ratio glycopyranose (product name: Isopropyl-beta-D-thiogalactopy, abbreviation: IPTG; manufacturer: UNI-ONWARD; Cat. No.: UR-IPTG-25G) and 1 gram of X-GAL (product name: 5-Bromo-4-chloro-3-indolyl β-D-galactopyranoside 98%, abbreviation: X-GAL; manufacturer: UNI-ONWARD; Cat. No.: UR-XGAL-2G), and pouring into a petri dish to cool and solidify after mixing. The titer determination method is as follows of: using 100, 1,000 and 10,000 times diluted eluate to infect 200 μL of log-phase of ER2738 E. coli, then adding it to 3 mL of top agar for mixing, and pouring it onto a solid LB culture dish to wait for solidification, continuously, incubating overnight under the conditions of 37° C. and 5% of CO₂, then using a shaker to mix the remaining eluate, 20 mL of LB medium and 200 μL of ER2738 E. coli at 200 rpm and incubating the reaction at 37° C. for 4.5 hours to amplify the selected phages, and finally purify the phages by means of PEG8000/NaCl precipitation method. The PEG8000/NaCl contains 20% (v/v) PEG8000 (product name: Poly (ethylene glycol); manufacturer: Sigma-Aldrich; Cat. No.: P5413-1KG) and 2.5M NaCl (product name: Sodium chloride; manufacturer: Honeywell fluka; Cat. No.: 31434S-1KG). The above steps are repeated for five rounds in total. In the fourth and fifth rounds, phage clones will be randomly selected for culture, and used in the subsequent enzyme-linked immunosorbent assay (ELISA) to screen out desired phages.

### <<Enzyme Combined Immunosorbent Assay Screening>>

In the “Enzyme Combined Immunosorbent Assay Screening”, the mouse CD80 recombinant protein is first fixed in the wells of a microplate (product name: Clear C-Shaped Immuno Nonsterile 96-Well Plates; manufacturer: Thermo Fisher Scientific; Cat. No.: 446612), and then the phage clones randomly selected in the fourth and fifth rounds of the “Phage Display Screening” are mixed with the diluent at a volume ratio of 1:2 to obtain a phage dilution, which is a phosphate buffered saline comprising 3% (v/v) bovine serum albumin (BSA) and 0.1% (v/v) polysorbate-20. Then add the phage dilution solution into the wells of the microplate and incubate at 25° C. for 1 hour to allow each phage to react with the human CD80 recombinant protein in the wells. After washing with washing solution, the Anti-M13 phage antibody with anti-wasabi peroxidase is added (product name: HRP/Anti-M13 Monoclonal Conjugate; manufacturer: GE Healthcare) and incubated at 25° C. for 1 hour, then wash again with washing solution right after that, 3,3′,5,5′-tetramethylbenzidine (TMB) is added as a chromogen, and continuously 3N hydrochloric acid (3N HCl) is added together to terminate the reaction. Constantly, a microplate reader is adopted to measure the absorbance at 450 nm to confirm whether the phages in each grid of the microplate are still bound to the mouse CD80 recombinant protein in the microplate. Among them, those still bound to the mouse CD80 recombinant protein in the microplate are called ELISA-positive phage colonies. Furthermore, the phagemids of the ELISA-positive phage colonies are collected and sequenced by Sanger sequencing to collect the DNA sequence of the phagemids, wherein the Sanger sequencing method is based on the ABI 3730XL system (product name: 3730xl DNA Analyzer; manufacturer: Applied Biosystem (ABI); Cat. No.: A41046). The washing solution described in “Enzyme Combined Immunosorbent Assay Screening” is phosphate buffered saline (PBS) containing 0.1% (v/v) polysorbate 20 (Tween 20). Talking to the experiment, based on the DNA sequence obtained by sequencing analysis, the two peptides obtained after completion of transcription and translation are the first peptide and the second peptide. Among them, the sequence of the first peptide is shown in SEQ ID NO: 1, and the second peptide is shown in SEQ ID NO: 2. Practically, the amino acid sequence of the first peptide is VLMGDPSVWWTY, and the amino acid sequence of the second peptide is YLIYEEEPLNSM. Accordingly, the first peptide and the second peptide are customized and synthesized from the Biomedical Translation Research Center of Academia Sinica and stored in a −20° C. environment.

### <<Preparation of Control Group and Pharmaceutical Composition of the Present Invention>>

For the present embodiment, the pharmaceutical composition of the present invention is a drug carrier coated with Doxorubicin and the surface itself includes polyethylene glycol (PEG) for modification. An example of using liposome is as a drug carrier and polyethylene glycol as a linker, and therefore the first peptide and/or the second peptide of the present invention are fixed on the surface of the drug carrier through the linker. The preparation steps are as follows of first, 8.5 mg (milligram, mg) of phosphoethanolamine (product name: DSPE-PEG-NHS; manufacturer: SUNBRIGHT; Cat. No.: DSPE-034GS) being added 250 μL of dimethylsulfoxide (product Name: Dimethyl Sulfoxide, BAKER ANALYZED™ ACS Reagent Grade; Manufacturer: J.T Baker; Cat. No.: 9224-03) to obtain a liposome material solution, in addition, taking another 250 μL of dichloromethane (Product Name: Dichloromethane; Manufacturer: Macron fine chemical; catalog number: 4881-08) to mixed with 3.1 mg of the first peptide or the second peptide to obtain a surface modification solution. After mixing 250 μL of liposome material solution and 250 μL of surface modification solution, 11 μL of triethylamine (product name: Triethylamine; manufacturer: J.T Baker; Cat. No.: W635-09) will be further added to obtain a mixed solution. Subsequently, the mixed solution is rotary evaporated with a rotary evaporator for 72 hours to obtain a mixture, which is then dissolved in phosphate buffered saline (PBS) and coated with a dialysis bag. The dialysis bag is thus soaked in phosphate buffered saline (PBS) for overnight dialysis. The molecular weight cutoff of the dialysis bag is 3,000 Da. After the dialysis is completed, the dialysate is freeze-dried in a freeze dryer for obtaining a dry product, which is stored in a −80° C. refrigerator before use. Afterward, 2.35 mg of dry matter and 1 mg of Doxorubicin (product name: Doxorubicin hydrochloride; manufacturer: J.T Baker; Cat. No.: 240214) are added to 10 mol of phospholipid (product name: LIPOID PC 18:0/18:0 (DSPC); manufacturer: Lipoid; Cat. No.: 816-94-4), and incubate at 60° C. for 1 hour to prepare suspension containing liposome-coated Doxorubicin. The suspension is purified and separated by means of a high-performance liquid chromatography tube (product name: high-performance liquid chromatograph, HPLC; manufacturer: Waters; model: 600E) filled with agarose gel (product name: sepharose 4B; manufacturer: Cytiva; Cat. No.: 17015001), and the proportion of each component in the suspension was quantified. The aforementioned peptide solution can be a first peptide solution or a second peptide solution. Therefore, depending on the type of peptide solution which is used, the surface of the liposome-coated Doxorubicin will be bound to the first peptide or the second peptide solution. Hereinafter, the doxorubicin-coated liposomes with the first peptide bound to the surface will be referred to as “LD-4-7”, and the liposome-coated Doxorubicin with the second peptide bound to the surface will be referred to as “LD-4-7”. LD-4-9”. The first peptide in LD-4-7 is connected to the surface of liposome-coated Doxorubicin via polyethylene glycol; the second peptide in LD-4-9 is cross-linked to the surface of liposome-coated Doxorubicin via polyethylene glycol. Additionally, in subsequent experiments, in order to test the effect of LD-4-7 and LD-4-9 of the present invention on cellular uptake, a control group was prepared. The control group prepared liposomes according to the aforementioned steps but did not add peptide solution. Therefore the liposomal surface of the control group does not include the first peptide or the second peptide of the present invention. The liposomes in the control group are hereinafter referred to as “LD-C”. The aforementioned liposomes are only examples of drug carriers, but the actual implementation is not limited thereto. The drug carriers may also be liposomes, micelle, nanoparticles, solid lipid nanoparticles, nanoemulsions (NE), or combinations thereof.

### <<Flow Cytometry Analysis>>

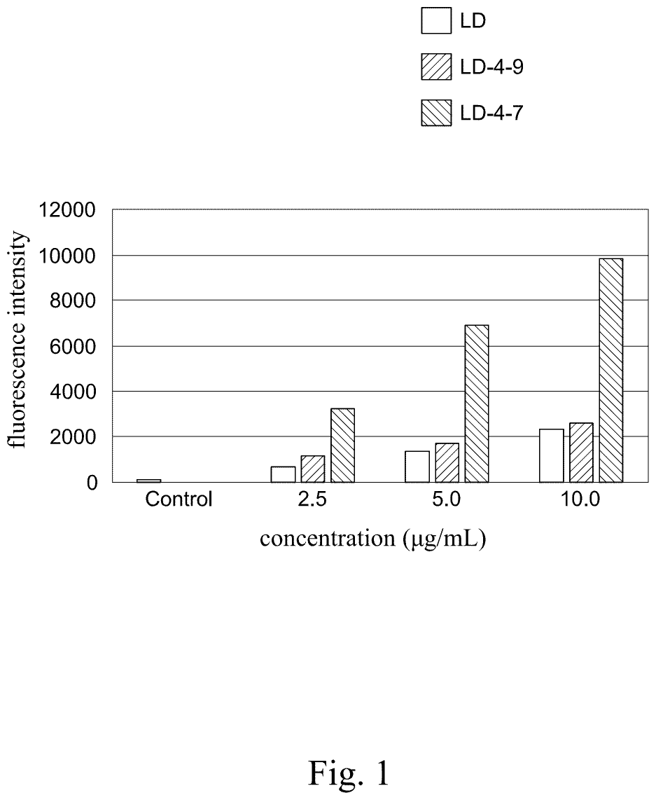

In order to confirm the effect of liposomes binding with different ligands for cellular uptake, for the embodiment, test cells are used and incubated with any one of LD-4-7, LD-4-9 and LD-C. After incubation, a flow cytometry is performed to detect fluorescence intensity. The test cells described in embodiment are HEK293T cells transfected with mouse CD80 gene. The open reading frame nucleic acid sequence is shown as SEQ ID NO: 3, and the nucleotide sequence of SEQ ID NO: 3 corresponding to the amino acid sequence is shown as SEQ ID NO: 4. The transfection method is to deliver the mouse CD80 cDNA ORF clone (product name: Cd80 (NM_009855.2) cDNA ORF clone, Mus musculus(house mouse)->NP_033985.3 Mus musculus CD80 antigen (Cd80), transcript variant 2, mRNA.; manufacturer: GenScript; Clone ID: OMu18782) into the test cells through in vitro DNA transfection reagent (product name: PolyJet™ In Vitro DNA Transfection Reagent; manufacturer: SignaGen; model: SL100688). Therefore, CD80 protein will be high expressed on the surface of the test cells. For the embodiment, 100,000 test cells are first seeded in each well of a 12-well cell culture plate (product name: Nunc™ Cell-Culture Treated Multidishes; manufacturer: Thermo Scientific™; catalog number: 150628) and then incubated at 37° C. overnight under 5% CO₂, and divided into groups according to the added liposomes. The liposomes are the aforementioned LD-4-7, LD-4-9 and LD-C. After adding the liposomes for each group, the liposomes of each group are incubated with the test cells for 8 hours at 37° C. and 5% CO₂. Then, each well is washed with phosphate buffered saline (PBS) as a washing buffer, and hence prepared as a pretreatment sample. The specific preparation steps are as follows. The drugs on the cell surface are removed by adding 0.1M glycine (pH 2.8) (product name: Glycine; manufacturer: Omics Bio; model: BT5031) for 10 minutes, and then using a cell separation reagent (product name: Accutase; manufacturer: Innovative Cell; model: AT104) separates the test cells from the 12-well cell culture plate to obtain a cell suspension. Furthermore, 1 mL of phosphate buffered saline is added, and the cell suspension is centrifugalized by a centrifugal machine. Immediately after removing the supernatant, again 1 mL of phosphate buffered saline is added for washing, the cell suspension is centrifugalized as well, and then the supernatant is removed. Finally, 400 μL of phosphate buffered saline is added to resuspend the test cells for obtaining a pretreatment sample. In this experiment/embodiment, the centrifugal force of the aforementioned centrifugal machine is 300 g, and the centrifugal time is 5 minutes. After completing the preparation of the pretreatment sample, a fluorescence spectrometer (product name: Multimode Plate Reader; manufacturer: Perkin Elmer; model: Enspire) or flow cytometer (product name: BD LSRFortessa™ Cell Analyzer; manufacturer: BD Biosciences; Cat. No.: 647794L6) is to analyze the pre-processed samples. The experimental results are shown in FIGS. 1 to 3 and Tables 1 to 3 below.

### <<Fluorescence Spectroscopy>>

Incubating the cells is the same method as above. After the culture period, the medium is removed and preparing it is for a pre-treatment sample. The specific preparation steps are as follows. 0.1 M of glycine (pH 2.8) (trade name: Glycine; manufacturer: Omics Bio; model: BT5031) is added for 10 minutes to remove drugs on the cell surface, and then 200 μL of 1% polyethylene glycol octylphenyl ether (product name: Triton™ X-100; manufacturer: Sigma-Aldrich; Cat. No.: T8787) is utilized to wash and lyse cells; adding 300 μl of isopropanol (product name: 2-Propanol; manufacturer: J.T. Baker; Cat. No.: 22D1861044) with 0.75N of hydrochloric acid. The solution is shaken for 30 minutes to extract Doxorubicin. After centrifugation at 12,000 rpm for 5 minutes, the pre-processed sample is analyzed with a fluorescence spectrometer. The excitation wavelength (λex) used by the fluorescence spectrometer is 488 nm, and the emission wavelength (λem) is 588 nm.

With reference to FIG. 1 and Table 1, which illustrate experimental results and graphs of fluorescence intensities of each group analyzed by a fluorescence spectrometer. The experimental results show that LD-4-7, LD-4-9 and LD-C are incubated with the test cells at different concentrations, and the fluorescence intensity results of the test cells are detected by the fluorescence spectrometer. In FIG. 1 and Table 1, the concentrations of liposomes added to each group are 2.5 μg/mL, 5.0 μg/mL, and 10.0 μg/mL respectively, and the experimental results of a group without liposomes are included as well. As a background value, the experimental results of the group without adding liposomes are presented in the column corresponding to “0 μg/mL” and the column corresponding to “LD-C” in Table 1. The higher the fluorescence intensity, which represents the test cells uptake more liposomes. The experimental results represented in FIG. 1 and Table 1 show that the groups using LD-4-7 and LD-4-9 detected at concentrations of 2.5 μg/mL, 5.0 μg/mL, and 10.0 μg/mL. The fluorescence intensities of groups using LD-4-7 and LD-4-9 are higher than that of the group using LD-C, and the fluorescence intensities of the group using LD-4-7 at the concentrations of 2.5 μg/mL, 5.0 μg/mL and 10.0 μg/mL are all higher than the group using LD-4-9, which means that the liposome binding with the first peptide of the present invention can indeed and effectively increase the liposome uptake by cells.

TABLE 1
The detected results (λex488/λem588) of the fluorescent intensities
of LD, LD-4-7 and LD-4-9 via the fluorescent spectrometer

| | Concentration | | | |
|---|---|---|---|---|
| Liposome | 0 μg/mL | 2.5 μg/mL | 5.0 μg/mL | 10.0 μg/mL |
| LD-C | 94 | 674 | 1334 | 2320 |
| LD-4-9 | — | 1144 | 1697 | 2600 |
| LD-4-7 | — | 3219 | 6900 | 9839 |

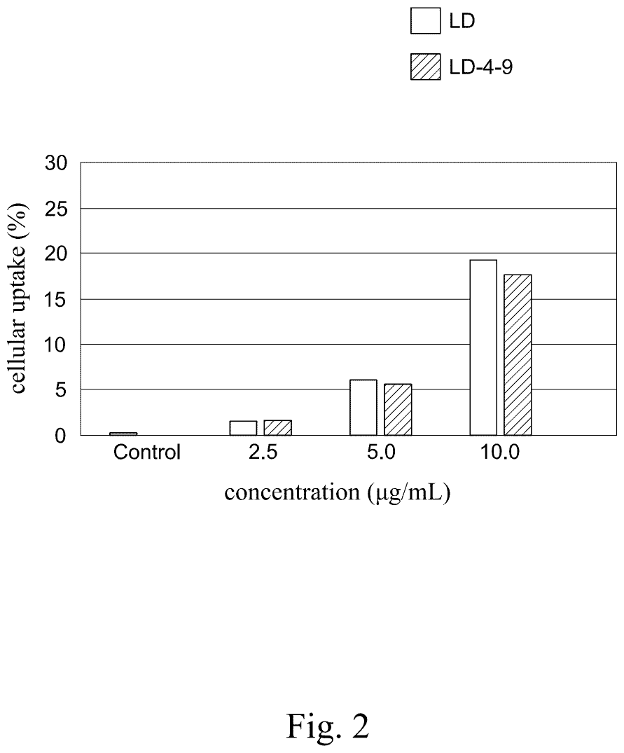

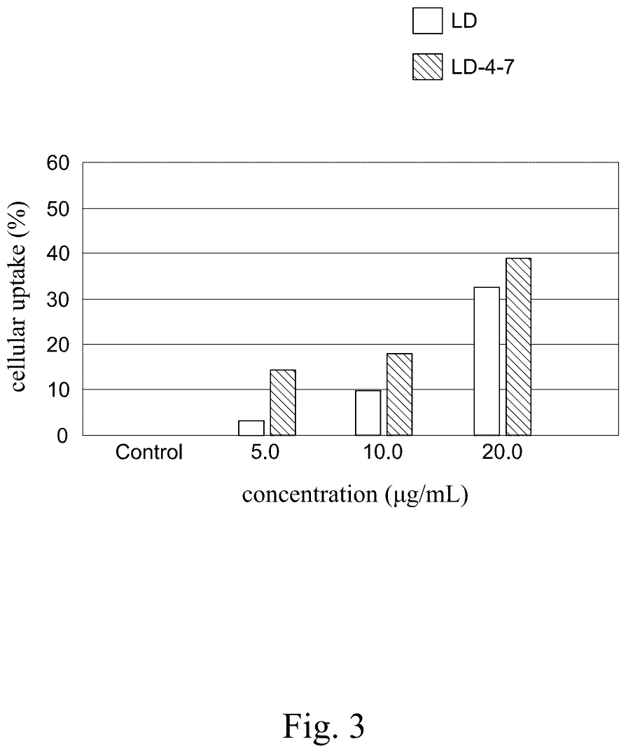

Besides, the present invention further monitors the status of cellular uptake, in which the proportion of cellular uptake is calculated by a flow cytometer (product name: BD LSRFortessa™ Cell Analyzer; manufacturer: BD Biosciences; Cat. No.: 647794L6) performing analysis. The calculation method is to use LD-C as the baseline fluorescence threshold, and the threshold and fluorescence channel according to the marker are set, in order to ensure the correct identification, the recording of the target cells and the elimination of background. Calculating it from all cell numbers for the proportion of cells that absorb fluorescence is done as well. The results of LD-C and LD-4-9 are shown in FIG. 2 and Table 2, and so does to the results of LD-C and LD-4-7 in FIG. 3 and Table 3 respectively. The concentrations of liposomes added to each group in FIG. 2 and Table 2 are 2.5 μg/mL, 5.0 μg/mL and 10.0 μg/mL respectively, and the experimental results of a group without adding liposomes are also included as a background value. The experimental results of the group without adding liposomes are presented in the column corresponding to “0 μg/mL” and in the row corresponding to “LD-C” in Table 2. The experimental results in FIG. 2 and Table 2 are shown LD-4-9 and LD-C are equally effective in promoting cellular uptake. The concentrations of liposomes added to each group in FIG. 3 and Table 3 are 5.0 μg/mL, 10.0 μg/mL and 20.0 μg/mL respectively, and the experimental results of a group without adding liposomes are included as another background value. The experimental results of the group without adding liposomes are presented in the column corresponding to “0 μg/mL” and in the row corresponding to “LD-C” in Table 3. The experimental results in FIG. 3 and Table 3 are shown if the added liposome concentrations are 5.0 μg/mL, 10.0 μg/mL and 20.0 μg/mL, wherein LD-4-7 performances are better than the control group in promoting cellular uptake. Accordingly, it can be seen from the aforesaid experimental results that LD-4-7 is able to promote immune macrophages with CD80 to take up more drugs, thereby achieving the technical efficiency of being a target drug.

TABLE 3
Experimental results of the cellular uptake
of LD-C and LD-4-7analyzed by flow cytometer

| | Concentration | | | |
|---|---|---|---|---|
| Liposome | 0 μg/mL | 5.0 μg/mL | 10.0 μg/mL | 20.0 μg/mL |
| LD-C | 0 | 4.4 | 13.2 | 43.9 |
| LD-4-7 | — | 19.4 | 24.2 | 52.5 |

As a conclusion, the present invention proves that the first peptide can indeed bind to the CD80 protein, and that binding the first peptide to liposomes does help cells to take up liposomes, and can be applied in drug delivery.

Although the present disclosure is disclosed in the foregoing embodiments, it is not intended to limit the present disclosure. Changes and modifications made without departing from the spirit and scope of the present disclosure belong to the scope of the claims of the present disclosure. The scope of protection of the present disclosure should be construed based on the following claims.

What is claimed is:

1. A novel peptide, which amino acid sequence is shown in SEQ ID NO: 1.

2. The novel peptide according to claim 1 being for binding to the CD80 protein.

3. A pharmaceutical composition comprising the novel peptide of claim 1 and a drug carrier, wherein the novel peptide is connected to a surface of the drug carrier.

4. The pharmaceutical composition according to claim 3, wherein the drug carrier is selected from the group consisting of: liposomes, microcells, nanoparticles, solid lipid nanoparticles, nanoemulsions, and combinations thereof.

5. The pharmaceutical composition according to claim 4, wherein the drug carrier is liposome.

6. The pharmaceutical composition according to claim 3, wherein the drug carrier contains a drug.

7. The pharmaceutical composition according to claim 6, wherein the drug is an anti-cancer drug.

8. The pharmaceutical composition according to claim 7, wherein the anti-cancer drug is Doxorubicin.

9. The pharmaceutical composition according to claim 3, wherein the novel peptide is connected to the surface of the drug carrier via a linker.

10. The pharmaceutical composition according to claim 9, wherein the linker is polyethylene glycol.


SEQUENCE LISTING (4 sequences)

SEQ ID NO: 1 - protein, 12 aa, synthetic construct
VLMGDPSVWWTY

SEQ ID NO: 2 - protein, 12 aa, synthetic construct
YLIYEEEPLNSM

SEQ ID NO: 3 - DNA, 921 bp, synthetic construct
  source 1..921: mol_type = other DNA
  CDS 1..921: protein_id = 4; translation = MACNCQLMQDTPLLKFPCPRLILLFVLLIRLSQVSSDVDEQLSK SVKDKVLLPCRYNSPHEDESEDRIYWQKHDKVVLSVIAGKLKVWPEYKNRTLYDNTTY SLIILGLVLSDRGTYSCVVQKKERGTYEVKHLALVKLSIKADFSTPNITESGNPSADT KRITCFASGGFPKPRFSWLENGRELPGINTTISQDPESELYTISSQLDFNTTRNHTIK CLIKYGDAHVSEDFTWEKPPEDPPDSKNTLVLFGAGFGAVITVVVIVVIIKCFCKHRS CFRRNEASRETNNSLTFGPEEALAEQTVFL
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttag

SEQ ID NO: 4 - protein, 306 aa, synthetic construct
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL